(19) United States
(12) Patent Application Publication
(10) Pub. No.: US 2018/0327514 A1
(43) Pub. Date: Nov. 15, 2018

(54) Functionalization of cellulose with lignin to produce high-value products

(71) Applicant: Teknologian tutkimuskeskus VTT Oy, Espoo (FI)

(72) Inventors: Arja Paananen, Espoo (FI); Geza Szilvay, Espoo (FI); Harri Setälä, Espoo (FI); Eva-Lena Hult, Espoo (FI); Hanna-Leena Alakomi, Espoo (FI)

(21) Appl. No.: 15/777,238

(22) PCT Filed: Nov. 25, 2016
(86) PCT No.: PCT/FI2016/050831

§ 371 Date: May 18, 2018

(30) Foreign Application Priority Data
Nov. 25, 2015 (FI) 20155871

(51) Int. Cl. C08B 11/12; A61L 15/28; A61L 15/44; C08H 7/00; C09D 101/28; C09D 197/00; C09J 101/28; C09J 197/00

(52) U.S. Cl. CPC C08B 11/12; A61L 15/28; A61L 15/44; C08H 6/00; A61L 2300/404; C09D 197/005; C09J 101/286; C09J 197/005; C09D 101/286

(57) ABSTRACT

The present invention concerns a process for the covalent attachment of lignin to cellulose in aqueous solutions, which process is characterized by preparing an aqueous mixture of lignin particles modified with tall oil fatty acids (TOFA), and reacting these TOFA-modified lignin particles with cellulose particles. The obtained modified cellulose particles are biodegradable, and have antimicrobial properties, whereby they are suitable for use in antibacterial textile surfaces (sportswear, medical textiles), tissue adhesives and as porous carriers in drug delivery. Further, the material is useful in high-volume products, such as adhesives and dispersants.

### BACKGROUND OF THE INVENTION

### Field of the Invention

The present invention concerns a process for the functionalization of cellulose with lignin to produce high-value lignin products that can be utilized as antimicrobial surfaces. Suitable end-products include e.g. antimicrobial wound dressings and antimicrobial adhesives or coatings (including paints).

Particularly, the process of the invention is carried out using covalent attachment of modified lignin particles to cellulose in aqueous solutions.

### Description of Related Art

Black liquor is the by-product from alkaline pulping processes, such as kraft and soda pulping, where most of the lignin is removed from the lignocellulosic feedstocks to free the cellulosic fibers for paper making.

Lignin is the main organic component in black liquor (25-30%). Currently, 98% of the lignin produced worldwide is used as a fuel, but there is a need to utilize the side-stream lignin in higher value products, and various, particularly environmental, possibilities are under extensive research.

One problem related to the environmental utilization of lignin is its low stability in aqueous dispersions. Currently its functionalization requires harsh conditions and usage of solvents. Among others, Hult et al. describes a solvent-based process for coating lignin onto paper board. However, the replacement of organic solvents with water would naturally be favoured.

Recently it has been shown that lignin can be processed into nanoparticles that enable handling of lignin as stable waterbased dispersions (Qian et al.). This is foreseen to solve the problem of low solubility and difficult processing of lignin, and consequently, to enhance the applicability of lignin. In the publication, it is, however, stated that formation of nanoparticles (NPs) requires acetylation of the lignin raw material. Also in WO 2006108637 A2 there is described a method for producing nanoparticles, but using lyophilization, which is a relatively harsh method.

When using lignin for functionalization, one advantageous option is to attach it to functionalized cellulose. Saastamoinen et al. and WO 2015011364 A2 both describe approaches for modifying the cellulose surface and turning it into a more hydrophobic form. Similarly, Aulin and Stroem describe oil-based coatings for use in packaging solutions. These processes, however, do not use lignin, although lignin would clearly provide a more environmental type of coating.

Recently, there have been studies utilizing tall oil fatty acid (TOFA) modified lignin in enhancement of barrier properties of paper boards (Hult et al.).

The use of lignin in functionalizations is also described in WO 2013050661 A1. However, this lignin is not used as nanoparticles, whereby its versatility and range of use is smaller, and its properties different.

### SUMMARY OF THE INVENTION

It is an object of the present invention to provide novel uses for side-stream lignin.

Particularly, it is an object of the present invention to provide functionalized antimicrobial products that utilize this lignin as an ecological raw-material.

The invention is defined by the features of the independent claims. Some specific embodiments are defined in the dependent claims.

According to a first aspect of the present invention, there is provided a process for the covalent attachment of lignin to cellulose in aqueous solutions.

According to a second aspect of the present invention, there is provided an antimicrobial wound dressing containing the lignin-modified cellulose of the invention.

According to a third aspect of the present invention, there is provided an antimicrobial coating or adhesive containing the lignin-modified cellulose of the invention.

Thus, the basic idea of the invention is the covalent attachment of lignin onto cellulose (optionally nanocellulose) in water-based solutions. This is facilitated by the production of a stable dispersion of NPs from lignin that has been modified using fatty acid mixtures (e.g. TOFA), and by the allylation of the cellulose, where double bonds in the TOFA and allyl groups in the cellulose serve as reactive functionalities for covalent linkage.

Considerable advantages are obtained by means of the invention. Lignin as nanoparticles solves the problem of utilizing lignin as stable water dispersions. TOFA-lignin can also be produced as nanoparticles, and this enables covalent functionalization of (nano)cellulose with lignin and utilization of lignin as functional additive or agent in water-based solutions. The TOFA-lignin NPs (TLNPs) can be easily mixed with other substances as they are stable monodisperse colloidal dispersions in water, and can hence be used as an additive in dispersions. The TLNP solutions can even endure freeze-drying, although it is preferred in the present embodiment to carry out the following process steps with the TLNPs in solution.

The produced TLNPs possess antimicrobial properties. Even though the antimicrobiality is not as strong as with silver NPs, the effect is significant, whereby this invention has application potential in replacing silver (or other metal) NPs in antimicrobial products, where metal NPs have disadvantages relating to both environmental and health factors. TLNP-nanocellulose mixture could also serve as an antimicrobial coating material, or as an additive in adhesives and paints.

Next, the invention will be described more closely with reference to the attached drawings and a detailed description.

### BRIEF DESCRIPTION OF THE DRAWINGS

FIG. 1 shows Scanning Electron Microscope (SEM) images of allylated cellulose surface (left) and TLNPs on allylated cellulose (right).

FIG. 2 is a graph of the antimicrobial activity of silver NPs (AgNPs) against Pseudomonas aeruginosa E-96726 cells, i.e. of the dose response to the antimicrobial agent (Minimum Inhibitory Concentration (MIC) 0.01 mg/ml).

FIG. 3 is a graphical presentation of the antimicrobial activity of TLNPs against S. aureus (FIG. 3A) and E. coli cells (FIG. 3B).

### EMBODIMENTS OF THE INVENTION

### Definitions

“Nanoparticles” (NPs) are in the context of the present invention intended to include particles having a diameter of less than 500 nm. In the present invention, mainly the lignin is used in nanosize, whereas the use of nanocellulose is optional. Typically, the length of the cellulose fibres is at least 1 μm, preferably 1-30 μm.

The “fatty acid mixtures” mentioned herein are intended to cover all mixtures containing at least 80% fatty acids by weight of the dry matter. Other typical components of these mixtures include rosin acids and unsaponifiables, although minor contents of other components are also possible. Particularly preferred in the present context are mixtures containing tall oil fatty acids (TOFA), as at least the main component.

It is to be understood that the embodiments of the invention disclosed are not limited to the particular process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

Thus, in one embodiment, the present invention concerns a process for the covalent attachment of lignin to cellulose in aqueous solutions, which process is characterized by preparing an aqueous mixture of lignin particles (typically nanoparticles) that have been modified with tall oil fatty acids (TOFA), and reacting these TOFA-modified lignin particles (TLNPs) with cellulose fibres or particles (including nanocrystals, CNC).

The main idea of this embodiment is the covalent attachment of lignin to cellulose, optionally in the form of nanocellulose, in water-based solutions. This is facilitated by the production of stable dispersion of nanoparticles (NP) from TOFA-modified lignin, and by the allylation of (nano)cellulose with allyl or 3-allyloxy-2-hydroxypropyl substituents, where the double bonds in the TOFA and the allyl groups in the cellulose serve as reactive functionalities for covalent linkage. The chemistry provided by TLNPs can also be utilized by using NPs as functional additive or agent in water based solutions.

In an embodiment of the present invention, the TLNPs are formed by esterifying the lignin using a mixture of natural fatty acids, preferably TOFA. According to a preferred embodiment, the esterification is carried out before the lignin NPs are formed.

TOFA is obtained from crude tall oil (CTO), a by-product of the pulping process, by separating and recovering the fatty acid rich fraction from the other components.

According to an embodiment of the invention, the used TOFA is a fatty acid mixture containing roughly 95 to 98% fatty acids, and 2 to 5% in total of saturated fatty acids and rosin acid and unsaponifiables. Of the total content of fatty acids, about 30 to 70% are C18:2 fatty acids and 40 to 60% are C18:1 fatty acids.

According to another embodiment of the invention, the hydroxyl groups of the lignin are esterified with the unsaturated fatty acids contained in the TOFA. These unsaturated fatty acids typically make up 80 to 99%, preferably 85 to 99%, by mass of the TOFA composition. Preferably, the content of the rosin acids in the TOFA is about 0.1 to 10%, and the content of unsaponifiables is less than 5%. Particularly preferred are compositions in which the total content of rosin acids and unsaponifiables is 5% or less.

The fatty acids of the used TOFA typically contain 16 to 20 carbon atoms, the main components being unsaturated C18 fatty acids, such as oleic and linoleic acids, having 1 and 2 double bonds, respectively (i.e. C18:1 and C18:2 fatty acids), and linolenic and pinolenic acid, both having three double bonds (i.e. C18:3 fatty acids).

Typically, of the fatty acids, the C18:3 fatty acids form up to 10%, whereas the main components are the fatty acids containing one and two double bonds. Typically, the content of C18:2 fatty acids is 20 to 70 parts by weight and the content of the C18:1 fatty acids is 20 to 70 parts by weight.

Although the present disclosure primarily relates to the use of TOFA compositions, it should be noted that other similar compositions of natural fatty acids, are also suitable for the present purpose. Thus, suitable fatty acid compositions can be obtained from various sources, such as rapeseed, linseed, hempseed oil, from soya bean, sunflower, colza, canola and olive oil; from mustard, palm, peanut, castor and coconut oil. Also oils from fish and seaweeds are in principle useful in the present context.

It may be necessary to submit said fatty acid sources to various treatment steps, including separation processing for example by distillation, in order to increase the ratio or purity of the fatty acid fractions, and obtain the fatty acid contents described above.

According to an embodiment, the acids, primarily fatty acids, and in particular unsaturated fatty acids, of the used fatty acid mixture, such as TOFA, are covalently linked to lignin to a varying degree of substitution. Typically, this degree of substitution is 40-100%, preferably 50-100%, most suitably about 50% (to provide the product TOFA L 50) or about 100% (to provide the product TOFA L 100).

According to an embodiment of the invention, the used lignin is selected from lignin that has been isolated from wood processing, typically extracted from softwood or hardwood pulp, preferably from kraft pulp, and most suitably using Lignoboost lignin. The chemical nature of any such lignin is affected by (i) the lignocellulosic source and (ii) the way the fibers of the source have been processed.

Esterification of the lignin, e.g. by fatty acids, can be carried out by a number of esterification processes known per se. The reaction can be carried out in a liquid medium or in dry phase. Examples of suitable method include transesterfication, esterification using reactive derivatives of the acid groups (e.g. acid anhydrides, acid chlorides), catalyzed direct esterification and the use of a screw reactor to avoid the use of a solvent.

A typical esterification is generally carried out at a temperature of 0 to 100° C. when operating at ambient pressure. Depending on the reactants a temperature of 0 to 50° C. can be particularly advantageous.

Conventional acid or alkaline catalysts can be employed.

In one embodiment, the esterification is carried out in a dry atmosphere, for example in an inert gas, such as nitrogen or argon. In another embodiment, esterification is carried out at reduced pressure, e.g. a pressure of 0.0001 to 0.1 bar absolute pressure.

The lignin esters (e.g. the TOFA-lignin) are isolated from the reaction mixtures, and are purified for example by washing, e.g. with ethanol, and are then dried gently, typically in a vacuum oven.

According to one option, the esterification is carried out by first converting the fatty acids mixture into the corresponding fatty acid chloride mixture and subsequently reacting it with lignin. The extent of the reaction can be adjusted by adjusting the ratio of lignin and TOFA.

As mentioned above, at least a part of the acids used for esterification contain unsaturation. The lignin, in turn, comprises phenolic hydroxyl groups, aliphatic hydroxyl groups or a combination thereof, in particular the hydroxyl groups comprise syringyl, guaiacyl or similar groups. By the present reaction, at least 40%, preferably 50 to 100% of the hydroxyl groups are substituted or esterified.

The active multiple double bond functionalities in TOFA enable further tailoring of the product properties.

For the preparation of the modified lignin NPs, the modified lignin are dissolved in a suitable organic solvent, such as THF, whereafter water is added, whereby the organic solvent can be evaporated to provide NPs in an aqueous solution.

When reacting the TOFA-esterified lignin according to the above embodiment with cellulose, a further advantage is obtained, whereby the compounding reaction can be carried out at low temperature, i.e. below 100° C., or e.g. at 65 or 80° C., even without added plasticizers.

The lignin that has been esterified using fatty acids, as described above, can either be used as such for antimicrobial purposes, or it can be adhered to a cellulose surface.

For use as such, the material is typically first separated from its aqueous mixture, whereby particles of fatty acid-modified lignin are obtained.

The cellulose raw material used in the reaction with the TOFA-lignin particles (TLNP) can be selected from any conventional plant cellulose, isolated e.g. from wood materials in a pulping process. Alternatively, bacterial cellulose can be used.

According to an embodiment of the invention, a suitable ratio of cellulose to TOFA-lignin is 10-60 mg cellulose/ml TOFA-lignin.

The modification of the cellulose, e.g. by allylation, prior to the reaction with the TLNP is not necessarily required. It has been found that the TLNPs are adhered also onto unmodified cellulose, but on cellulose that has been modified e.g. by allylation, the modified lignin provides a more even coverage. Our results show that TLNPs are evenly distributed on allylated cellulose, whereas NPs are attached as big clusters on a reference cellulose sample.

We have shown that the herein described TLNP's possess antimicrobial properties. This property brings up the application potential of TLNPs in replacement of silver (or other) NP's, that are known to have both health and environmental risks. Thus, potential application areas could for example be antimicrobial paints, sportswear and medical textiles (for example tissue adhesives and plaster coatings).

Another option for utilization of these antimicrobial properties could be the use of TLNPs grafted on nanocellulose as a coating material, i.e. applying an antimicrobial layer on the surface instead of attaching NP's to the whole bulk material.

Due to its suitable hydrophobicity and other beneficial properties, the TLNP's could also serve as an additive in adhesives and paints.

Thus, the most potential applications are both in high-volume products, such as adhesives and dispersants, and in more challenging applications, like biodegradable antibacterial textile surfaces (sportswear, medical textiles), tissue adhesives and as porous carriers in drug delivery.

The following non-limiting examples are intended merely to illustrate the advantages obtained with the embodiments of the present invention.

### EXAMPLES

### Example 1—Preparation of Tofa-Lignin NPs

For the preparation of the Tofa-lignin NPs, tall oil fatty acid modified lignin samples TOFA-L-50 and TOFA-L-100 were dissolved in THF (step 1). Milli Q water was slowly added to the solution (step 2). Thereafter, the THF was evaporated using a rotavapor (step 3). After this the samples where dialyzed against water for 3 days using cellulose acetate dialysis membrane (MWCO 1000 Da). Two different batches were prepared by altering the time for solvent exchange.

The obtained product particles (TLNP50 and TLNP100) were analyzed by dynamic light scattering (DLS) and shown to have the characteristics listed in Table 1. ‘Size’ describes the size distribution of particles in solution and ‘Zeta’ the corresponding zeta potential. The dimensions obtained by atomic force microscopy (AFM) were in the same range as with DLS for batch 1 samples (Table 2).

TABLE 1
TLNP samples from Batches 1 and 2.

| TLNP50 | | | | TLNP100 | | | |
|---|---|---|---|---|---|---|---|
| Batch 1 | Size | 140 ± 50 | nm | Batch 1 | Size | 160 ± 50 | nm |
| | Zeta | −29 ± 8 | mV | | Zeta | −30 ± 8 | mV |
| Batch 2 | Size | 295 ± 150 | nm | Batch 2 | Size | 420 ± 200 | nm |
| | Zeta | −36 ± 7 | mV | | Zeta | −36 ± 8 | mV |

TABLE 2
TLNP samples from Batch 1.

| | Diameter (nm) | Height (nm) |
|---|---|---|
| TLNP50 | 70-190 | 55-160 |
| TLNP100 | 50-200 | 50-175 |

### Example 2—Modification of Cellulose Fibres with TOFA-Lignin Derivatives

Reaction Procedure:

Tall Oil Fatty Acid Derivative of Lignin

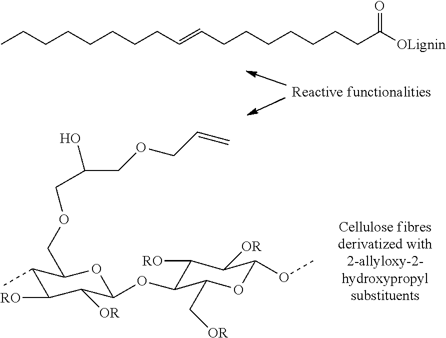

Two samples were prepared 1) 250 ml of TOFA-lignin-50 NPs (TLNP50) or 2) TOFA-lignin-100 NPs (TLNP100) (from the previous example) was added into a 500 ml reactor. 1) 5 g or 2) 10 g of modified cellulose fibres (Domsjö, enzyme-treated, allylated) were added, respectively.

The reaction mixtures were stirred at a rate of 350 rpm, and heated up to 65° C. After one hour, 0.25 g of ammonium persulfate (APS) was added in 5 ml of water. The reaction mixtures were then stirred overnight (for about 16-18 h) at 65° C.

The reaction mixture was cooled down to 22° C. The cellulose fibres with TLNPs were filtrated through filter paper (S&S 595) using a Büchner funnel and washed several times with deionized water.

The product samples, TOFA-lignins on cellulose (TOFA-lignin-50-5 g-cellulose/TOFA-L50-5C and TOFA-lignin-100-10 g-cellulose/TOFA-L100-10C), were dried at room temperature overnight and weighed.

The yields were 92% of TOFA-L50-5C and 106% of TOFA-L100-10C.

When analyzing the products in a solution of 30% water in acetone→no colour in the solution even after several days. This indicates that most probably covalent bonds are formed between allylated cellulose and TOFA-lignin

A hydrolysis test with 30% of 0.1 M NaOH solution in acetone, in turn→rather soon a brown colour formed into liquid phase. This is an indication of the hydrolysis of ester bonds between TOFA and lignin.

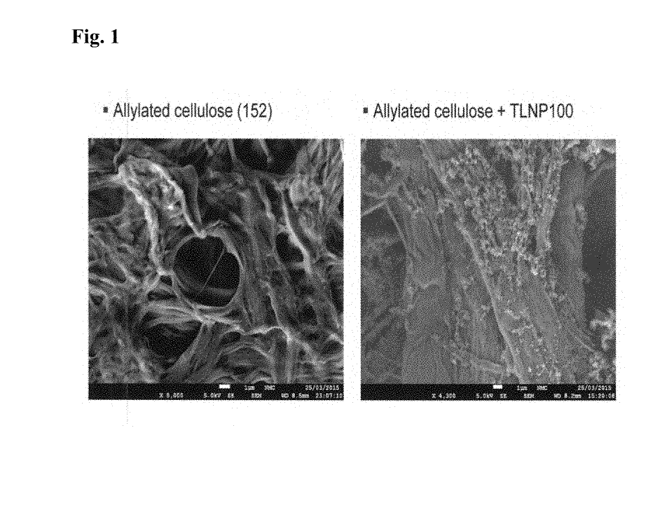

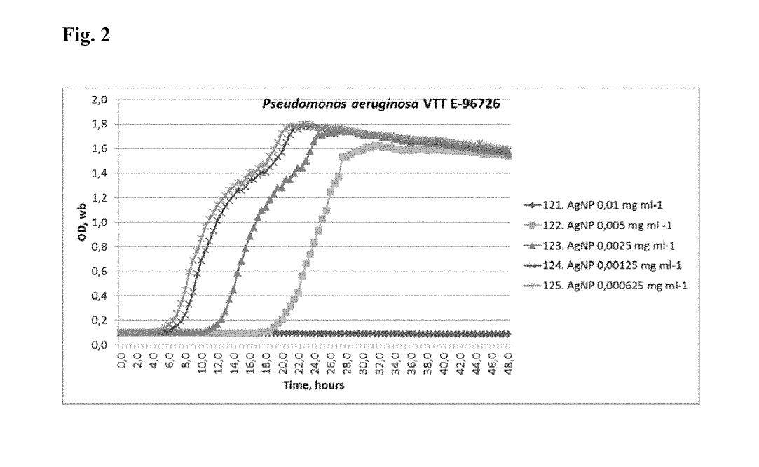

Further, SEM images (FIG. 1) showed the manner in which the TOFA100-modified lignin particles covered the cellulose surface.

### Example 3—Examination of the Antimicrobial Properties of TOFA-Modified Lignin Nanoparticles in Solution and Cellulose Samples Coated with TOFA-Modified Lignin

The antimicrobial activity of test samples was analysed with modified CLSI M100-S19 method in Mueller-Hinton II broth. Two-fold dilutions from the test samples were prepared into broth and mixed with overnight grown bacterial inoculum. Growth of the samples at 37° C. was monitored with automated turbidometer, Bioscreen for 48 hours. Minimal inhibitory concentration (MIC) values and growth inhibition % values were calculated.

The tested samples included the conventional silver nanospheres (Sigma) used in several antimicrobial products, as well as the TOFA-lignin particles of the present invention (TLNP50 and TLNP100). The stock concentrations used in this example are listed in the following Table 3.

TABLE 3
Stock concentrations

| Sample | Concentration (mg/ml) |
|---|---|
| Ag nanospheres (average size 20 nm) | 0.02 |
| TLNP50 (average size 140 nm) | 1 |
| TLNP50 (average size 295 nm) | 1 |
| TLNP100 (average size 160 nm) | 1 |
| TLNP100 (average size 420 nm) | 1 |

The microbes used in the analysis are listed in the following Table 4.

TABLE 4
Microbes

| Target microbes | Strain code |
|---|---|
| Escherichia coli | VTT E-94564 |
| Staphylococcus aureus | VTT E-70045 |
| Pseudomonas aeruginosa | VTT E-96726 |

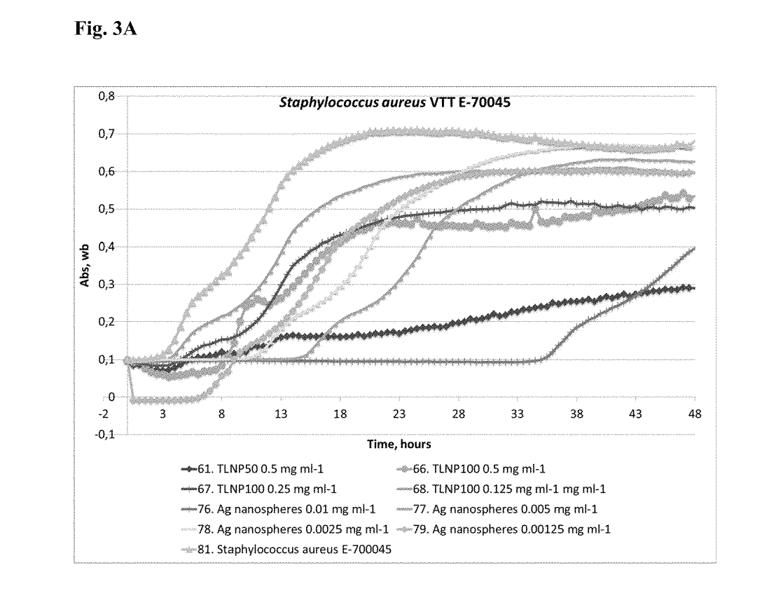

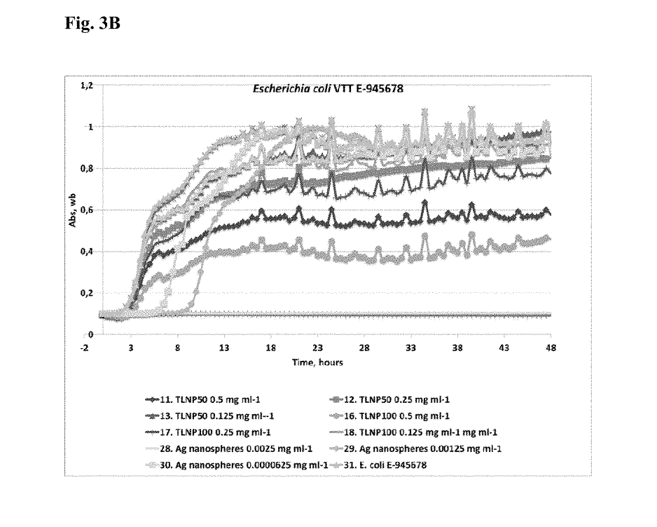

The results of the analyses are shown in FIG. 2 (for silver only) and in FIG. 3 where antimicrobial activity of silver nanoparticles, and TLNP50 (295 nm) and TLNP100 (420 nm) are presented as examples, respectively. The activity as growth inhibitions (%) of all studied samples are shown in Table 5. As the results show, all the examined samples had antimicrobial activity.

Gram positive Staphylococcus aureus appeared to be more sensitive than Gram negative target microbes to lignin-NP samples. For Lignin-NP samples the minimum inhibitory concentration (MIC) values higher than the highest examined concentration.

TABLE 5
Antimicrobial activity of TLNPs as growth inhibition % (compared
to the growth of control without antimicorbials)

| | Growth inhibition (%) with target microbes | | | | | |
|---|---|---|---|---|---|---|
| | Staphylococcus aureus | | Escherichia coli | | Pseudomonas aeruginosa | |
| | VTT E-70045 | | VTT E-945678 | | VTT E-96726 | |
| | Test concentration | | Test concentration | | Test concentration | |
| | mg/ml | | mg/ml | | mg/ml | |
| Sample | 0.5 | 0.25 | 0.5 | 0.25 | 0.5 | 0.25 |
| TLNP50 (140 nm) | 21 | 12 | 30 | 10 | 24 | 17 |
| TLNP50 (265 nm) | 66 | Nd | 41 | 15 | Nd | Nd |
| TLNP100 (160 nm) | 22 | 15 | 25 | 12 | 24 | 15 |
| TLNP100 (420 nm) | 32 | 30 | 57 | 23 | Nd | Nd |

Nd = no data

The Antimicrobial activity of TLNP100 modified cellulose samples was examined against Staphylococcus aureus VTT E-70045 by applying target cells directly on the sample surface. Briefly, overnight grown cells were diluted and 10⁵ cells applied on test pieces (diameter 12 mm). Samples were incubated at 37° C. for one hour and viability of the cells analyzed with plate count technique. The results (colony forming units/sample) are shown in Table 6. On reference surfaces (filter paper) the cells survived during the incubation period.

TABLE 6
Survival of target microbes (colony forming units per sample)
on tested sample surfaces after one hour contact time.

| | Target microbe | |
|---|---|---|
| | Staphylococcus aureus | Escherichia coli |
| Sample | VTT E-70045 | VTT E-945678 |
| Filter paper | 2.2 × 10³ | 1 × 10⁴ |
| Unmodified cellulose (APS) | 2.0 × 10² | 5 × 10³ |
| Allylated cellulose (APS) | <10 | <10 |
| TLNP100 on Unmodified | <10 | <10 |
| cellulose (APS) | | |
| TLNP100 on Allylated | <10 | <10 |
| cellulose (APS) | | |

### Example 4—Examination of the Antimicrobial Properties of TOFA-Lignin-Modified Cellulose Films

TOFA-lignin nanoparticles (TLNP50 and TLNP100) were prepared using THF as a solvent. 0.48 g of TOFA-L-50 or TOFA-L-100 was dissolved in 360 ml of THF. 480 g of Milli Q water was slowly (30 min) added to the solution under stirring at rt. Thereafter, the main part of THF was evaporated using rotavapor, and finally the TLNP suspension was dialysed using a membrane with cut-off 3500 Da. The final concentration of lignin nanoparticles in water suspension was 1 mg/ml.

250 ml of these TLNP-50 and TLNP-100 nanoparticle suspensions were used for each functionalization batch and added into a 500 ml reactor. 3.7 g of cellulose fibres was added, and stirred at 350 rpm. The reaction mixture was heated up to +65° C., and 0.25 g of ammonium persulfate (APS) in 5 ml of water was added. The stirring was continued for 18 hrs at +65° C. The reaction mixture was then cooled down to +22° C. The cellulose fibres with TLNPs were filtrated onto a filter paper (ϕ 125 mm, S&S 595) using a Büchner funnel, washed several times with deionized water, and dried at RT.

Reference filter paper sheets with 3.7 g cellulose fibres were prepared in similar manner as described above using APS as an initiator only in 250 ml of deionized water.

The antimicrobial activity of the thus prepared TOFA-lignin nanoparticle solutions (TLNP50 and TLNP100) were analysed with modified CLSI M100-S19 method in Mueller-Hinton II broth. Silver nanospheres (Sigma-Aldrich 795925, average size 10 nm) were used as reference. Escherichia coli VTT E-94564, Staphylococcus aureus VTT E-70045 and Pseudomonas aeruginosa VTT E-96726 used as target microbes were obtained from VTT Culture Collection. Briefly, two-fold dilutions from the test samples were prepared into broth and mixed with an inoculum (10⁶ cells ml⁻¹) prepared from overnight at 37° C. grown bacterial cells. Growth of the samples in microwell system at 37° C. was monitored with automated turbidometer, Bioscreen C™ (Thermo Scientific, Finland) and Research Express software (Transgalactic Ltd, Finland) for 48 hours. Growth inhibition % values were calculated from the growth curves.

The antimicrobial activity of these modified cellulose films was examined against Staphylococcus aureus VTT E-70045 and Escherichia coli VTT E-94564 by applying target cells directly on the sample surface. Filter paper, unmodified cellulose and a commercial silver blaster were used as reference. Briefly, overnight in Trypticase soy broth grown cells were diluted in peptone saline and 10⁵ cells applied on test pieces (diameter 12 mm). Samples were incubated at 37° C. for 30 minutes and viability of the cells analyzed with plate count technique on Plate Count agar.

The results are shown in the following Table 7.

TABLE 7
Summary of the antimicrobial activity of the samples
as growth inhibition (%) after 48 hour incubation

| | Staphylococcus | | Escherichia | | Pseudomonas | |
|---|---|---|---|---|---|---|
| | aureus | | coli | | aeruginosa | |
| | VTT E-70045 | | VTT E-945678 | | VTT E-96726 | |
| | Test | | Test | | Test | |
| | concentration | | concentration | | concentration | |
| | mg ml⁻¹ | | mg ml⁻¹ | | mg ml⁻¹ | |
| Sample | 0.5 | 0.25 | 0.5 | 0.25 | 0.5 | 0.25 |
| TLNP50 | 51 ± 21 | 16 ± 1 | 25 ± 10 | 12 ± 2 | 21 ± 4 | 13 ± 6 |
| (140 nm) | | | | | | |
| TLNP100 | 31 ± 6 | 17 ± 9 | 39 ± 24 | 15 ± 1 | 12 ± 1 | 3 ± 1 |
| (160 nm) | | | | | | |
| AgNP** | 64 ± 16 | 28 ± 3 | 97 ± 1 | 91 ± 1 | 97 ± 0 | 93 ± 2 |

As becomes evident from the results, TLNP-50 and TLNP-100 samples at 0.5 mg ml⁻¹ concentration had antimicrobial activity, growth inhibition % against S. aureus E-70045 was 51±21 and 31±6, respectively (Table 7). Antimicrobial activity of the samples was weaker against Gram-negative target microbes E. coli and P. aeruginosa than against Gram-positive S. aureus cells.

Allylated cellulose and lignin particle functionalized cellulose surfaces had antimicrobial activity and during 30 min exposure cells applied on the surface died (Table 8). During the experiment cells survived on the filter paper, unmodified cellulose and silver plaster. This indicates fast interaction of the target cells with allylated cellulose and functionalized cellulose surface.

TABLE 8
Survival of target microbes (colony forming units per sample)
on tested sample surfaces after 30 min contact time.

| | Staphylococcus aureus | Escherichia coli |
|---|---|---|
| Sample | VTT E-70045 | VTT E-94564 |
| Filter paper | 5 × 10³ | 1.0 × 10⁴ |
| Unmodified cellulose (APS) | 2.0 × 10⁴ | 5.0 × 10³ |
| Silver blaster | 2.0 × 10⁴ | 1.0 × 10⁴ |
| Allylated cellulose (APS) | <10 | <10 |
| TLNP100 on unmodified | <10 | <10 |
| cellulose (APS) | | |
| TLNP100 on allylated | <10 | <10 |
| cellulose (APS) | | |

### INDUSTRIAL APPLICABILITY

The present material can be used as biodegradable antibacterial textile surfaces (sportswear, medical textiles), tissue adhesives and as porous carriers in drug delivery, i.e., more generally, for replacement of conventional antibacterial surfaces. Further, the present material is useful in high-volume products, such as adhesives and dispersants.

### CITATION LIST

### Patent Literature

WO 2006108637 A2
WO 2013050661 A1
WO 2015011364 A2

### Non-Patent Literature

Hult, E.-L., Ropponen, J., Poppius-Levlin, K., Ohra-Aho, T., Tamminen, T., Industrial Crops and Products (2013), 50, 694-700
Qian, Y., Deng, Y., Qiu, X., Li, H., Yang, D., Green Chemistry (2014), 16(4), 2156-2163
Saastamoinen, P., Mattinen, M.-L., Hippi, U., Nousiainen, P., Sipila, J., Lille, M., Suurnakki, A., Pere, J., BioResources (2012), 7(4), 5749-5770
Aulin, C. and Stroem, G., Industrial & Engineering Chemistry Research (2013), 52(7), 2582-2589

1. A process for the covalent attachment of lignin to cellulose in aqueous solutions comprising preparing an aqueous mixture of lignin particles, modifying these particles with fatty acids, and reacting the obtained fatty acid-modified lignin particles with cellulose fibers or particles.

2. The process of claim 1, wherein the lignin is selected from isolated lignin, extracted from softwood or hardwood pulp.

3. The process of claim 1, wherein the lignin is selected from lignin nanoparticles (NPs), preferably having a diameter of less than 500 nm, more preferably of 50-300 nm, and most suitably of 70-200 nm.

4. The process of claim 1, wherein the fatty acids used to modify the lignin contain, or are selected from, tall oil fatty acids (TOFA).

5. The process of claim 1, wherein the fatty acids used to modify the lignin are selected from fatty acid mixtures, e.g. tall oil fatty acids (TOFA), containing 80-99%, preferably 85-99%, by weight of unsaturated fatty acids.

6. The process of claim 1, wherein the fatty acids used to modify the lignin are selected from fatty acid mixtures, e.g. TOFA, containing rosin acids in an amount of 0.1-10%, preferably ≤5%, by weight, and most suitably containing a total content of rosin acids and unsaponifiables of ≤5% by weight.

7. The process of claim 1, wherein the fatty acids used to modify the lignin are selected from fatty acid mixtures, e.g. TOFA, containing mainly fatty acids having 16 to 20 carbon atoms, the main components preferably being unsaturated C18 fatty acids, and most suitably being oleic acid (C18:1), linoleic acid (C18:2), linolenic acid and/or pinolenic acid (both C18:3).

8. The process of claim 1, wherein the fatty acids used to modify the lignin are selected from fatty acid mixtures, e.g. TOFA, containing mainly fatty acids including up to 10% by weight of C18:3 fatty acids, 20-70% by weight of C18:2 fatty acids, and 20-70% by weight of C18:1 fatty acids.

9. The process of claim 1, wherein the fatty acids used to modify the lignin, e.g. the TOFA, are converted into the corresponding fatty acid chloride mixture prior to the reaction with the lignin.

10. The process of claim 1, wherein the modification reaction is targeted for a degree of substitution, by fatty acids in the lignin, of 40-100%, preferably 50-100%, most suitably about 50% or about 100%.

11. The process of claim 1, wherein the aqueous mixture has been achieved by adding water to a solution of fatty acid-modified lignin in a suitable organic solvent, e.g. tetrahydrofuran (THF), and evaporating the organic solvent.

12. The process of claim 1, wherein the cellulose has been allylated prior to the reaction.

13. The process of claim 1, wherein the reaction between the fatty acid-modified lignin and the cellulose takes place in an aqueous solution, and with the help of a radical initiator, such as ammonium persulfate (APS).

14. The process of claim 1, wherein the reaction between the fatty acid-modified lignin and the cellulose takes place at a temperature of <100° C., preferably a temperature of about 65 or 80° C.

15. An antimicrobial wound dressing comprising a conventional dressing material containing a fatty acid-lignin-cellulose prepared by a process for the covalent attachment of lignin to cellulose in aqueous solutions, the process comprising preparing an aqueous mixture of lignin particles, modifying these particles with fatty acids, and reacting the obtained fatty acid-modified lignin particles with cellulose fibers or particles.

16. The antimicrobial wound dressing of claim 15, impregnated with a solution containing 1-10 mg/ml of said fatty acid-lignin-cellulose.

17. An antimicrobial coating or adhesive comprising a coating mixture or adhesive mixture containing 1-10 mg/ml of a fatty acid-lignin-cellulose prepared by a process for the covalent attachment of lignin to cellulose in aqueous solutions, the process comprising preparing an aqueous mixture of lignin particles, modifying these particles with fatty acids, and reacting the obtained fatty acid-modified lignin particles with cellulose fibers or particles.

18. (canceled)

19. The antimicrobial coating or adhesive of claim 17, wherein the modified lignin particles have a degree of substitution, by fatty acids in the lignin, of 40-100%, preferably 50-100%, most suitably about 50% or about 100%.

20. The process of claim 1, wherein the lignin is selected from kraft pulp or Lignoboost lignin.

21. The process of claim 1, wherein the lignin is selected from lignin nanoparticles (NPs) having a diameter of 50-300 nm.
